(19) United States
(12) Patent Application Publication
(10) Pub. No.: US 2024/0217932 A1
(43) Pub. Date: Jul. 4, 2024

(54) PYRIDIUM ESTERS

(71) Applicant: The Procter & Gamble Company, Cincinnati, OH (US)

(72) Inventors: Brandon L. Mash, Pittsburgh, PA (US); Sean Nicholas Natoli, Liberty Township, OH (US); Gregory Scot Miracle, Liberty Township, OH (US)

(21) Appl. No.: 18/521,144

(22) Filed: Nov. 28, 2023

(30) Foreign Application Priority Data
Nov. 28, 2022 (EP) 22209905.3

(51) Int. Cl. C07D 213/55; A01N 25/04; A01N 25/30; A01N 25/34; A01N 43/40; A01P 1/00

(52) U.S. Cl. CPC C07D 213/55; A01N 25/04; A01N 25/30; A01N 25/34; A01N 43/40; A01P 1/00

(57) ABSTRACT

A pyridinium ester according to Formula I

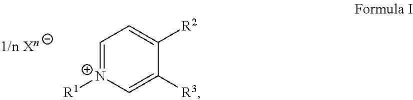

wherein 1/n Xⁿ⁻ is a counterion;
wherein n is 1 or 2;
wherein one of R² and R³ is H and the other is selected from the group consisting of Formula II, Formula III and Formula IV

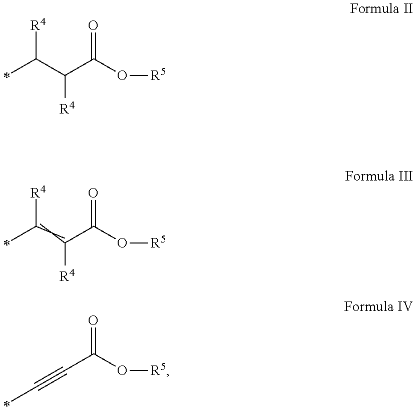

wherein * represents the point of attachment to the heteroaryl ring,
wherein Formula III is selected from the group consisting of a Z-configuration, an E-configuration, and mixtures thereof, preferably an E-configuration, wherein when R² is H, R¹ is a branched or unbranched, saturated or unsaturated, C₄-C₁₂ hydrocarbyl group;
when R³ is H, R¹ is a branched or unbranched, saturated or unsaturated, C₁-C₁₂ hydrocarbyl group,
and
wherein each R⁴ is independently selected from hydrogen and a C₁-C₄ hydrocarbyl group
and
wherein R⁵ is a branched or unbranched, saturated or unsaturated, C₄-C₁₂ hydrocarbyl group.

### TECHNICAL FIELD

The present disclosure relates to pyridinium esters and compositions comprising the esters.

### BACKGROUND

Antibacterial household compositions are becoming more widely used with a focus on kitchen, bath and toilet cleaning. In addition to the sanitization or disinfection the compositions are required to clean and/or provide shine to the treated surface. Although there exists a number of antimicrobial treatment compositions, there is a desire to find more efficient and more environmentally friendly compositions. Furthermore, the development of isotropic formulations with gentle, biodegradable actives is desirable.

There is therefore the need for an antibacterial treatment composition that has a good environmental and human safety profile, that shows strong antibacterial efficacy, that can be used on food contact surfaces and that shows good cleaning, even on tough greasy soils, and/or leaves the surfaces shiny and without streaks.

### SUMMARY

According to the first aspect of the present disclosure, there is provided a pyridinium ester according to claim 1.

According to the second aspect of the present disclosure, there is provided an antimicrobial composition comprising the pyridinium ester of the present disclosure.

According to the third aspect of the present disclosure, there is provided an article treated with the composition of the second aspect of the present disclosure. The article is preferably in the form of a disposable or partially reusable substrate comprising one or more nonwoven layers. The article provides sanitization to surfaces, in particular hard surfaces. The article is sometimes herein referred to as “the article of the present disclosure”.

According to the fourth aspect of the present disclosure, there is provided a method of sanitizing a surface using the composition of the present disclosure. There is also provided the use of the composition of the present disclosure to provide antimicrobial benefits to a surface, in particular to an inanimate surface.

The elements of the of the present disclosure described in relation to any of the aspects of the present disclosure apply mutatis mutandis to the other aspects of the present disclosure.

### DETAILED DESCRIPTION

The present disclosure relates to pyridinium esters and compositions comprising the pyridinium esters. The composition provides antimicrobial benefits. The composition comprises an antimicrobial system. The system comprises three components that combined provide a very effective antimicrobial action. The composition is preferably an aqueous composition comprising at least 50% by weight of the composition of water. The composition is especially suitable for treating hard surfaces.

As used herein, the phrase “chain atoms” means the sum of all atoms in an indicated group or moiety, excluding hydrogen atoms. The chain atoms may be in a linear constitution, a branched constitution, and/or a ring constitution.

As used herein, “hydrocarbyl group” means any univalent radical, derived from a hydrocarbon.

All percentages, ratios and proportions used herein are by weight percent of the composition, unless otherwise specified. All average values are calculated “by weight” of the composition, unless otherwise expressly indicated. All ratios are calculated as a weight/weight level, unless otherwise specified.

All measurements are performed at 25° C. unless otherwise specified.

Unless otherwise noted, all component or composition levels are in reference to the active portion of that component or composition, and are exclusive of impurities, for example, residual solvents or by-products, which may be present in commercially available sources of such components or compositions.

### Pyridinium Ester

The pyridinium ester of the present disclosure has the following formula (Formula I)

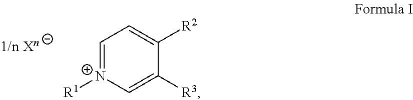

wherein 1/n Xⁿ⁻ is a counterion;
wherein n is 1 or 2;
wherein one of R² and R³ is H and the other is selected from the group consisting of Formula II, Formula III and Formula IV,

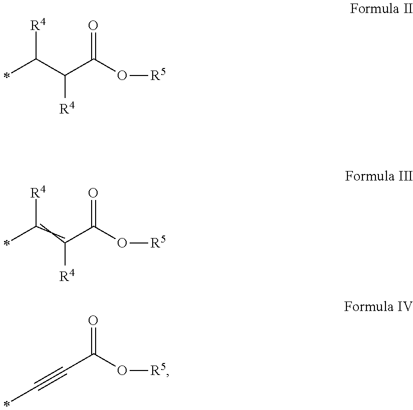

wherein * represents the point of attachment to the heteroaryl ring,
Formula III can have an E- or Z-configuration, Formula III can be selected from the group consisting of a Z-configuration, an E-configuration, and mixtures thereof, preferably an E-configuration,
wherein when R² is H, R¹ is a branched or unbranched, saturated or unsaturated, C₄-C₁₂ hydrocarbyl group, preferably C₄-C₁₀, more preferably C₄-C₈ and especially C₆ hydrocarbyl group;
when R³ is H, R¹ is a branched or unbranched, saturated or unsaturated, C₁-C₁₂ hydrocarbyl group, preferably C₂-C₁₀, more preferably C₄-C₈ and especially C₆ hydrocarbyl group; and
wherein each R⁴ is independently selected from hydrogen and a C₁-C₄ hydrocarbyl group and
wherein R⁵ is a branched or unbranched, saturated or unsaturated, C₄-C₁₂ hydrocarbyl group, preferably R⁵ is a branched or unbranched C₆-C₈ saturated hydrocarbyl group, more preferably a C₆ hydrocarbyl group.

Pyridinium esters wherein one of R₂ and R₃ is H and the other is Formula III, provide good antimicrobial properties when used as part of the antimicrobial system comprising a surfactant and a solvent. Preferred pyridinium esters are those in which one of R₂ and R₃ is H and the other is Formula III, wherein all R₄ are H, and wherein R₁ and R₅ are independently selected from a branched or unbranched C₆-C₈ saturated hydrocarbyl groups, preferably R₁ and R₅ are independently selected from unbranched C₆ saturated hydrocarbyl groups.

Preferably, Formula III is

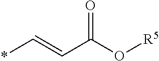

More preferably R5 is a C₆-C₈ hydrocarbyl group, especially C₆H₁₃.

Preferably, the pyridinium ester is selected from the group consisting of:

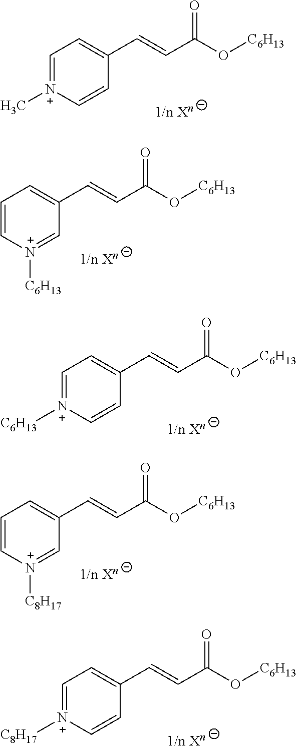

Especially preferred are the pyridinium esters are selected from the group consisting of:

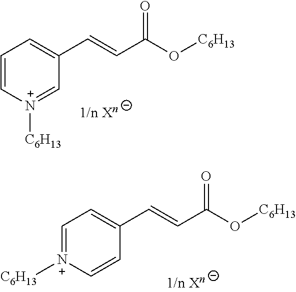

and mixtures thereof.

Mixtures of the pyridinium esters of the present disclosure can also be good in terms of antimicrobial properties when part of the antimicrobial system of the present disclosure.

The pyridinium esters of the present disclosure can be obtained by reacting pyridine carboxaldehydes with malonic acid mono-esters in the presence of pyridine and amino acids such as alanine as catalysts. The resulting compounds are modified by adding hydrocarbon moieties both to the pyridine nitrogen atom and to the terminal carboxylate oxygen atom. Hydrocarbon moieties are selected to increase the overall hydrophobicity of the resulting compounds, since this is believed to improve antimicrobial efficacy.

An example is illustrated herein below:

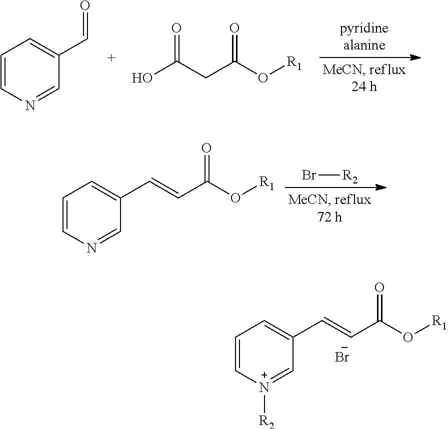

Examples of starting materials suitable for preparing the pyridinium esters of the present disclosure, in particular alkyl pyridinium esters include, but are not limited to, Ethyl (2E)-3-(3-pyridinyl)-2-propenoate (prepared as described in: Synlett (2011), (12), 1723-1726); 4-Pyridinepropanoic acid, ethyl ester (Prepared as described in: Journal of the American Chemical Society (2011), 133(3), 582-594); Ethyl (2E)-3-(4-pyridinyl)-2-propenoate (synthesized according to the procedure described in: Organic Letters (1999), 1(4), 579-582); Ethyl 3-pyridinepropanoate (Prepared as disclosed in: Bioorganic & Medicinal Chemistry (2009), 17(5), 2047-2068); Ethyl 3-(3-pyridinyl)-2-propynoate (prepared from the acid, the preparation of which is given in the Journal of Heterocyclic Chemistry (1981), 18(3), 519-23, according to: Zhurnal Organicheskoi Khimii (1976), 12(2), 443-8); Ethyl (2Z)-3-(3-pyridinyl)-2-propenoate (prepared as described in: Tetrahedron Letters (2018), 59(1), 14-17); and Ethyl 3-(4-pyridinyl)-2-propynoate (prepared as described in: WO2002/060438 A1).

Examples of starting materials suitable for preparing the pyridinium esters of the present disclosure, in particular α-alkylated alkyl pyridinium esters include, but are not limited to, Ethyl 2-(3-pyridinylmethylene)butanoate, Ethyl 2-(4-pyridinylmethylene)butanoate, Ethyl α-(1-methylethyl)-4-pyridinepropanoate, Ethyl α-(1-methylethyl)-3-pyridinepropanoate, Ethyl α-ethyl-4-pyridinepropanoate, and Ethyl α-ethyl-3-pyridinepropanoate (available for purchase from Aurora Fine Chemicals, LLC. San Diego, CA, USA); Methyl (2Z)-2-methyl-3-(4-pyridinyl)-2-propenoate and 2-Propenoic acid, 2-methyl-3-(3-pyridinyl)-, ethyl ester, (2Z)-(prepared as disclosed in: Chemistry—A European Journal (2013), 19(45), 15281-15289); Ethyl (2E)-2-methyl-3-(3-pyridinyl)-2-propenoate (prepared as disclosed in: Chemistry—A European Journal (2009), 15(18), 4538-4542), which upon hydrogenation yields Ethyl α-methyl-4-pyridinepropanoate; and Ethyl (2E)-2-methyl-3-(4-pyridinyl)-2-propenoate (prepared as disclosed in: ChemPlusChem (2016), 81(8), 893-898), which upon hydrogenation yields Ethyl α-methyl-4-pyridinepropanoate.

Examples of starting materials suitable for preparing the pyridinium esters of the present disclosure, in particular β-alkylated alkyl pyridinium esters include, but are not limited to, Ethyl (2E)-3-(3-pyridinyl)-2-butenoate, Ethyl β-methyl-3-pyridinepropanoate, Ethyl (2Z)-3-(3-pyridinyl)-2-butenoate, Ethyl (2Z)-3-(4-pyridinyl)-2-butenoate, Ethyl (2E)-3-(4-pyridinyl)-2-butenoate, Methyl β-ethyl-3-pyridinepropanoate, Methyl β-ethyl-4-pyridinepropanoate, Methyl β-propyl-3-pyridinepropanoate, Methyl 3-(4-pyridinyl)-2-butenoate, Ethyl β-ethyl-4-pyridinepropanoate, Ethyl β-ethyl-3-pyridinepropanoate, Ethyl β-propyl-3-pyridinepropanoate, Ethyl β-(1-methylpropyl)-4-pyridinepropanoate, and Ethyl β-(1-methylpropyl)-3-pyridinepropanoate (all available for purchase from Aurora Fine Chemicals, LLC. San Diego, CA, USA); Ethyl β-methyl-4-pyridinepropanoate (available for purchase from Enamine US Inc., Monmouth Junction, NJ, USA); Ethyl 3-(4-pyridinyl)-2-butenoate (prepared as disclosed in WO 2001/085714 A1); 3-Pyridinepropanoic acid, β-propyl-, ethyl ester, (βS)-; 4-Pyridinepropanoic acid, β-propyl-, ethyl ester, (βS)-; 3-Pyridinepropanoic acid, β-2-methylpropyl-, ethyl ester, (βS)-; and 4-Pyridinepropanoic acid, β-2-methylpropyl-, ethyl ester, (βS)-(all prepared as disclosed in: ACS Catalysis (2022), 12(4), 2434-2440); and Methyl (2E)-3-(3-pyridinyl)-2-hexenoate and Methyl (2Z)-3-(3-pyridinyl)-2-hexenoate (prepared as disclosed in: Chemical Communications (Cambridge, United Kingdom) (2020), 56(7), 1101-1104).

Examples of starting materials suitable for preparing the pyridinium esters of the present disclosure, in particular α,β-alkylated alkyl pyridinium esters include, but are not limited to, Ethyl (2Z)-2-methyl-3-(3-pyridinyl)-2-butenoate; 2-Butenoic acid, 2-methyl-3-(3-pyridinyl)-, ethyl ester; Ethyl 2-methyl-3-(4-pyridinyl)-2-butenoate; Ethyl 2-ethyl-3-(3-pyridinyl)-2-butenoate; and Ethyl 2-ethyl-3-(4-pyridinyl)-2-butenoate (all available for purchase from Aurora Fine Chemicals, LLC. San Diego, CA, USA); and Ethyl (2E)-2-methyl-3-(3-pyridinyl)-2-butenoate (available for purchase from Enamine US Inc., Monmouth Junction, NJ, USA).

Any of the starting esters listed above can be converted to an ester of an alcohol R⁵-OH via a simple transesterification reaction, wherein R⁵ is a branched or unbranched, saturated or unsaturated, C₄-C₁₂ hydrocarbyl group. The pyridinium esters of the present disclosure may then be prepared through quaternization of the pyridine nitrogen via typical nucleophilic displacement of a suitable leaving group X from a compound such as R¹-X, wherein R¹ is a branched or unbranched, saturated or unsaturated, C₁-C₁₂ or C₄-C₁₂ hydrocarbyl group.

Examples of suitable counterion X (charge balancing anions to form the salts) include but are not limited to: fluoride, chloride, bromide, iodide, perchlorate, hydrogen sulfate, sulfate, aminosulfate, nitrate, dihydrogen phosphate, hydrogen phosphate, phosphate, bicarbonate, carbonate, methosulfate, ethosulfate, cyanate, thiocyanate, tetrachlorozincate, borate, tetrafluoroborate, acetate, chloroacetate, cyanoacetate, hydroxyacetate, aminoacetate, methylaminoacetate, di- and tri-chloroacetate, 2-chloro-propionate, 2-hydroxypropionate, glycolate, thioglycolate, thioacetate, phenoxyacetate, trimethylacetate, valerate, palmitate, acrylate, oxalate, malonate, crotonate, succinate, citrate, methylene-bis-thioglycolate, ethylene-bis-iminoacetate, nitrilotriacetate, fumarate, maleate, benzoate, methylbenzoate, chlorobenzoate, dichlorobenzoate, hydroxybenzoate, aminobenzoate, phthalate, terephthalate, indolylacetate, chlorobenzenesulfonate, benzenesulfonate, toluenesulfonate, biphenyl-sulfonate and chlorotoluenesulfonate. Those of ordinary skill in the art are well aware of different counterions which can be used in place of those listed above. Preferably, the charge balancing anion have a molecular weight below 200, counterions suitable for the pyridinium esters of the present disclosure are listed in the table herein below. Specially preferred for use herein are halides, in particular chloride and bromide.

| Anion Name | Empirical Formula | MW |
|---|---|---|
| Methanesulfonate | CH₃SO₃ | 95.1 |
| Ethanesulfonate | CH₃CH₂SO₃ | 109.12 |
| Benezenesulfonate | C₆H₅SO₃ | 157.17 |
| p-Toluenesulfonate | CH₃C₆H₄SO₃ | 171.19 |
| Cumenesulfonate | C₉H₁₁O₃S | 199.25 |
| Xylenesulfonate | (CH₃)₂C₆H₃SO₃ | 185.22 |
| Chloride | Cl | 35.45 |
| Bromide | Br | 79.90 |

The pyridinium esters of the present disclosure are very resilient in terms of pH, i.e., they maintain their antimicrobial properties across a broad pH range. The pyridinium esters of the present disclosure present a good environmental profile.

### Antimicrobial Treatment Composition

The composition of the present disclosure is suitable to be used on surfaces, preferably on inanimate surfaces, more preferably on hard surfaces. The composition can be delivered onto the surface, by for example spraying the composition, followed by wiping the surface, preferably without rinsing or by using a substrate, such as a wipe impregnated with the composition of the present disclosure. The composition provides good cleaning and/or shine to the treated surface. The composition can be a concentrated composition, that can be diluted before use or a ready to use composition. Preferably, the composition is a ready-to-use sprayable composition.

As used herein, the terms “microbe” or “microbial” should be interpreted to refer to any of the microscopic organisms studied by microbiologists or found in the use environment of a treated surface. Such organisms include, but are not limited to, bacteria and fungi as well as other single-celled organisms such as mould, mildew and algae. Viruses (enveloped and non-enveloped) and other infectious agents are also included in the term microbe.

“Antimicrobial” further should be understood to encompass both microbiocidal and microbiostatic properties. That is, the term includes microbe killing, leading to a reduction in number of microbes, as well as a retarding effect of microbial growth, wherein numbers may remain more or less constant (but nonetheless allowing for slight increase/decrease).

For case of discussion, this description uses the term antimicrobial to denote a broad-spectrum activity (e.g. against bacteria and fungi, or against bacteria and viruses). When speaking of efficacy against a particular microorganism or taxonomic rank, the more focused term will be used (e.g. antifungal to denote efficacy against fungal growth in particular). Using the above example, it should be understood that efficacy against fungi does not in any way preclude the possibility that the same antimicrobial composition may demonstrate efficacy against another class of microbes.

By “hard surface”, it is meant herein hard surfaces found in households, especially domestic households. Surfaces to be cleaned include kitchens and bathrooms, e.g., floors, walls, tiles, windows, cupboards, sinks, showers, shower plastified curtains, wash basins, WCs, fixtures and fittings and the like made of different materials like ceramic, vinyl, no-wax vinyl, linoleum, melamine, glass, steel, kitchen work surfaces, any plastics, plastified wood, metal or any painted or varnished or sealed surface and the like. Household hard surfaces also include household appliances including, but not limited to refrigerators, freezers, washing machines, automatic dryers, ovens, microwave ovens, dishwashers and so on. Such hard surfaces may be found both in private households as well as in commercial, institutional and industrial environments.

The compositions herein are aqueous compositions, comprising at least 50% by weight of the composition of water, preferably from 80% to 95% and more preferably from 90% to 96% by weight of the composition of water. Preferably, the compositions of the present disclosure are clear compositions. Without wishing to be bound by theory, it is believed that in order to provide the best antimicrobial efficacy, the antimicrobial system should be in solution. The composition should preferably be in the form of a clear, isotropic liquid.

The compositions of the present disclosure preferably can be non-thickened, or water like, having a viscosity of from 1 mPa·s to 5 Pa·s, or can be thickened, having a viscosity of from 50 Pa·s to 1200 Pa·s, more preferably 100 Pa·s to 800 Pa·s, most preferably 200 Pa·s to 600 Pa·s when measured at 20° C. with a AD1000 Advanced Rheometer from Atlas® shear rate 10 s⁻¹ with a coned spindle of 40 mm with a cone angle 2° and a truncation of ±60 μm.

### Antimicrobial System

The composition of the present disclosure comprises an antimicrobial system comprising a pyridinium ester. The pyridinium ester needs only be present in germicidal effective amounts, which can be as little as 0.001% to less than 10% by weight of the composition. In more preferred compositions, the cleaning composition comprises the pyridinium ester, or a mixture thereof, at a level of from from about 0.0025 to about 5%, preferably from 0.005% to 1.5% by weight of the composition.

A germicidal effective amount of the antimicrobial agent typically results in at least a log 4 reduction of gram-negative bacteria, using the method of EN13697 (Chemical Disinfectants Bactericidal Activity Testing), in 5 minutes.

### Surfactant

The composition of the present disclosure preferably comprises surfactants, more preferably from 0.01% to 10%, preferably from 0.05% to 8%, more preferably from 0.08% to 5%, more preferably 0.1% to 3% by weight of the composition of surfactant. The surfactant also contributes to the cleaning and/or shine provided by the composition.

### Alkoxylated Alcohol Nonionic Surfactants

Suitable alkoxylated alcohol nonionic surfactants are according to the formula RO-(A)ₙH, wherein: R is a primary C₄ to C₁₈, preferably a C₆ to C₁₆, more preferably a C₁₀ to C₁₆ and even more preferably from C₁₂ to C₁₄ branched or linear alkyl chain, or a C₆ to C₂₈ alkyl benzene chain; A is an ethoxy or propoxy or butoxy unit, or mixtures thereof, and wherein n is an integer from 1 to 30, preferably from 1 to 15, more preferably from 3 to 12 even more preferably from 3 to 8. Preferred R chains for use herein are the C₁₀ to C₁₆ linear or branched alkyl chains. Especially preferred for use herein is an alcohol alkoxylated having a C₁₂-C₁₄ chain length and having from 8 to 10 ethoxy groups.

Suitable branched alkoxylated alcohols may be selected from the group consisting of: C₄-C₁₀ alkyl branched alkoxylated alcohols, and mixtures thereof. The branched alkoxylated alcohol can be derived from the alkoxylation of C₄-C₁₀ alkyl branched alcohols selected form the group consisting of: C₄-C₁₀ primary mono-alcohols having one or more C₁-C₄ branching groups.

By C₄-C₁₀ primary mono-alcohol, it is meant that the main chain of the primary mono-alcohol has a total of from 4 to 10 carbon atoms. The C₄-C₁₀ primary mono-alcohol can be selected from the group consisting of: methyl butanol, ethyl butanol, methyl pentanol, ethyl pentanol, methyl hexanol, ethyl hexanol, propyl hexanol, dimethyl hexanol, trimethyl hexanol, methyl heptanol, ethyl heptanol, propyl heptanol, dimethyl heptanol, trimethyl heptanol, methyl octanol, ethyl octanol, propyl octanol, butyl octanol, dimethyl octanol, trimethyl octanol, methyl nonanol, ethyl nonanol, propyl nonanol, butyl nonanol, dimethyl nonanol, trimethyl nonanol and mixtures thereof.

The C₄-C₁₀ primary mono-alcohol can be selected from the group consisting of: ethyl hexanol, propyl hexanol, ethyl heptanol, propyl heptanol, ethyl octanol, propyl octanol, butyl octanol, ethyl nonanol, propyl nonanol, butyl nonanol, and mixtures thereof. Preferably the C₄-C₁₀ primary mono-alcohol is selected from the group consisting of: ethyl hexanol, propyl hexanol, ethyl heptanol, propyl heptanol, and mixtures thereof.

The C₄-C₁₀ primary mono-alcohol is most preferably ethyl hexanol, and propyl heptanol. Especially preferred for use herein are ethoxylated ethyl hexanol comprising from 4 to 10 ethoxy groups.

In the branched alkoxylated alcohol, the one or more C₁-C₄ branching group can be substituted into the C₄-C₁₀ primary mono-alcohol at a C1 to C3 position, preferably at the C1 to C2 position, more preferably at the C2 position, as measured from the hydroxyl group of the starting alcohol.

The branched alkoxylated alcohol can comprise from 1 to 14, preferably from 2 to 7, more preferably from 4 to 6 ethoxylate units, and optionally from 1 to 9, preferably from 2 to 7, more preferably from 4 to 6 of propoxylate units.

The branched alkoxylated alcohol is preferably 2-ethyl hexan-1-ol ethoxylated to a degree of from 4 to 6, and propoxylated to a degree of from 4 to 6, more preferably, the alcohol is first propoxylated and then ethoxylated. Another preferred branched alkoxylated alcohols are 2-alkyl-1-alkanols such as alkoxylated C₁₀ guerbet alcohols with 1 to 14, preferably from 2 to 7, more preferably from 3 to 6 ethoxylate or ethoxylate-propoxylate units.

Non-limiting examples of suitable branched alkoxylated alcohols are, for instance, Ecosurf® EH3, EH6, and EH9, commercially available from DOW, and Lutensol® XP alkoxylated Guerbet alcohols & Lutensol® XL ethoxylated Guerbet alcohols available from BASF.

Linear alcohol alkoxylated nonionic surfactants preferred herein are alkoxylated nonionic surfactants with a C₈, C₁₀, C₁₂, mixtures of C₈ to C₁₀, mixtures of C₁₀ to C₁₂, mixtures of C₉ to C₁₁ linear alkyl chain and 8 or less ethoxylate units, preferably 3 to 8 ethoxylate units.

Non-limiting examples of suitable linear alkoxylated nonionic surfactants for use herein are Dobanol® 91-2.5 (R is a mixture of C₉ and C₁₁ alkyl chains, n is 2.5), Dobanol® 91-5 (R is a mixture of C₉ to C₁₁ alkyl chains, n is 5); Dobanol® 91-10 (R is a mixture of C₉ to C₁₁ alkyl chains, n is 10); Greenbentine DE60 (R is a C10 linear alkyl chain, n is 6); Marlipal 10-8 (R is a C₁₀ linear alkyl chain, n is 8); Neodol 91-8 (R is a mixture of C₉ to C₁₁ alkyl chains, n is 8); Empilan® KBE21 (R is a mixture of C₁₂ and C₁₄ alkyl chains, n is 21); Lutensol ON30 (R is C₁₀ linear alkyl chain, n is 3); Lutensol ON50 (R is C₁₀ linear alkyl chain, n is 5); Lutensol ON70 (R is C₁₀ linear alkyl chain, n is 7); Novel 610-3.5 (R is mixture of C₆ to C₁₀ linear alkyl chains, n is 3.5); Novel 810FD-5 (R is mixture of C₈ to C₁₀ linear alkyl chains, n is 5); Novel 10-4 (R is C₁₀ linear alkyl chain, n is 4); Novel 1412-3 (R is mixture of C₁₂ to C₁₄ linear alkyl chains, n is 3); Lialethl® 11-5 (R is a C₁₁ linear alkyl chain, n is 5); Lialethl® 11-21 (R is a mixture of linear and branched C₁₁ alkyl chain, n is 21), or mixtures thereof.

The alkoxylated nonionic surfactant may be a secondary alcohol ethoxylate such as for example the Tergitol™-15-S surfactants having the general formula shown below and commercially available from DOW

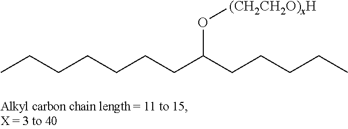

Tergitol 15-S surfactants Preferred secondary alcohol ethoxylate surfactants have 3-9 EO units.

Another suitable alkoxylated nonionic surfactant is an alkyl ethoxy alkoxy alcohol, preferably wherein the alkoxy part of the molecule is propoxy, or butoxy, or propoxy-butoxy. More preferred alkyl ethoxy alkoxy alcohols are of formula (II):

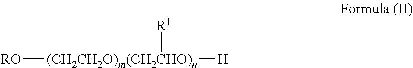

wherein:
R is a branched or unbranched alkyl group having 8 to 16 carbon atoms;
R¹ is a branched or unbranched alkyl group having 1 to 5 carbon atoms;
n is an integer from 1 to 10; and m is an integer from 6 to 35.

R is preferably from 12 to 15, preferably 13 carbon atoms. R¹ is preferably a branched alkyl group having from 1 to 2 carbon atoms. n is preferably an integer from 1 to 5. m is preferably an integer from 8 to 25. Preferably, the weight average molecular weight of the ethoxylated alkoxylated nonionic surfactant of formula (II) is from 500 to 2000 g/mol, more preferably from 600 to 1700 g/mol, most preferably 800 to 1500 g/mol.

The ethoxylated alkoxylated nonionic surfactant can be a polyoxyalkylene copolymer. The polyoxyalkylene copolymer can be a block-heteric ethoxylated alkoxylated nonionic surfactant, though block-block surfactants are preferred. Suitable polyoxyalkylene block copolymers include ethylene oxide/propylene oxide block polymers, of formula (III):

(EO)ₓ(PO)_{y}(EO)ₓ, or

(PO)ₓ(EO)_{y}(PO)ₓ Formula III

wherein EO represents an ethylene oxide unit, PO represents a propylene oxide unit, and x and y are numbers detailing the average number of moles ethylene oxide and propylene oxide in each mole of product. Such materials tend to have higher molecular weights than most non-ionic surfactants, and as such can range between 1000 and 30000 g/mol, although the molecular weight should be above 2200 and preferably below 13000 to be in accordance with the present disclosure. A preferred range for the molecular weight of the polymeric non-ionic surfactant is from 2400 to 11500 Daltons. BASF (Mount Olive, N.J.) manufactures a suitable set of derivatives and markets them under the Pluronic trademarks. Examples of these are Pluronic (trademark) F77, L62 and F88 which have the molecular weight of 6600, 2450 and 11400 g/mol respectively.

Other suitable ethoxylated alkoxylated nonionic surfactants are described in Chapter 7 of Surfactant Science and Technology, Third Edition, Wiley Press, ISBN 978-0-471-68024-6.

Most preferably the alkoxylated nonionic surfactant is selected from the group consisting of: 2-propylheptyl EO8 (Lutensol XL89-BASF); 2-propylheptyl EO5 (Lutensol XL50-BASF); C₁₀ alcohol EO5 (Lutensol ON 50-BASF); C₁₀-alcohol EO7 (Lutensol ON 70-BASF); C₈-C₁₀ EO5 (Novel 810 FD5 Sasol); C₁₀ EO4 (Novel 10-4 Sasol); Tergitol 15-S-3; Tergitol 15-S-5; Tergitol 15-S-7; and Ethyl hexanol PO5EO6 (Ecosurf EH6-Dow). These surfactants have surprisingly been found to potentiate the antibacterial efficacy of the pyridinium esters.

### Alkyl Polyglucosides

Alkyl polyglycosides are biodegradable nonionic surfactants which are well known in the art and can be used in the compositions of the present disclosure. Suitable alkyl polyglycosides can have the general formula CₙH₂ₙ₊₁O(C₆H₁₀O₅)ₓH wherein n is preferably from 8 to 16, more preferably 8 to 14, and x is at least 1. Examples of suitable alkyl polyglucoside surfactants are the TRITON™ alkyl polyglucosides from Dow; Agnique PG, Disponil APG and Glucopon alkyl polyglucosides from BASF. Preferred alkyl polyglucoside surfactants are those where n is 8 to 12, more preferably 8 to 10, such as for example Triton™ CG50 (Dow). These surfactants have surprisingly been found to enhance the antimicrobial efficacy of the pyridinium esters.

### Alkyl Amine Oxide

Suitable amine oxide surfactants include: R₁R₂R₃NO wherein each of R₁, R₂ and R₃ is independently a saturated or unsaturated, substituted or unsubstituted, linear or branched hydrocarbyl chain having from 1 to 30 carbon atoms. Preferred amine oxide surfactants are amine oxides having the following formula: R₁R₂R₃NO wherein R1 is a hydrocarbyl chain comprising from 1 to 30 carbon atoms, preferably from 6 to 20, more preferably from 8 to 16 and wherein R₂ and R₃ are independently saturated or unsaturated, substituted or unsubstituted, linear or branched hydrocarbyl chains comprising from 1 to 4 carbon atoms, preferably from 1 to 3 carbon atoms, and more preferably are methyl groups. R1 may be a saturated or unsaturated, substituted or unsubstituted linear or branched hydrocarbyl chain.

Highly preferred amine oxides are C₈ dimethyl amine oxide, C₁₀ dimethyl amine oxide, C₁₂ dimethyl amine oxide, C₁₄ dimethyl amine oxide, and mixtures thereof C₈ dimethyl amine oxide is commercially available under the trade name Genaminox® OC from Clariant; C₁₀ dimethyl amine oxide is commercially available under the trade name Genaminox® K-10 from Clariant; C₁₂ dimethyl amine oxide is commercially available under the trade name Genaminox® LA from Clariant and of Empigen OB from Huntsman; C₁₄ amine oxide is commercially available under the trade name of Empigen OH 25 from Huntsman. Other suitable amine oxide surfactants are cocoyldiethoxy amine oxide available under the trade name of Genaminox CHE from Clariant, and cocamydopropyl amine oxide commercially available under the trade name of Empigen OS/A from Huntsman. Particularly preferred amine oxide surfactants are C₁₀ dimethyl amine oxide such as Genaminox K-10. These surfactants have surprisingly been found to greatly enhance the antibacterial efficacy of the pyridinium esters.

### Alkyl Glucamide Surfactants

The composition of the present disclosure may comprise an alkyl glucamide surfactant. Glucamide surfactants are non ionic surfactants in which the hydrophilic moiety (an amino-sugar derivative) and the hydrophobic moiety (a fatty acid) are linked via amide bonds. This results in a chemical linkage, which is highly stable under alkaline conditions. Particularly preferred alkyl glucamide surfactants are N-alkyl-N-acylglucamides of the formula (IV):

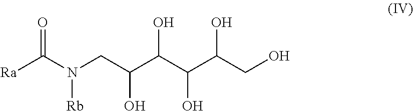

wherein Ra is a linear or branched, saturated or unsaturated hydrocarbyl group having 6 to 22 carbon atoms, and Rb is a C₁-C₄ alkyl group. Particularly preferably, Rb in formula (III) is a methyl group. Non-limiting examples of these glucamide surfactants are: N-octanoyl-N-methylglucamide, N-nonanoyl-N-methylglucamide, N-decanoyl-N-methylglucamide, N-dodecanoyl-N-methylglucamide, N-cocoyl-N-methylglucamide, available under the trade name of GlucoPure Foam from Clariant, N-lauroyl/myristoyl-N-methylglucamide, (available under the trade name of GlucoPure Deg from Clariant, and N-octanoyl/decanoyl-N-methylglucamide, available under the trade name of GlucoPure Wet by Clariant.

Alkyl glucamine surfactants are suitable for the composition of the present disclosure.

These surfactants are described in EP16184415 and US20190055496.

### Zwitterionic and Amphoteric Surfactants

The hard surface cleaning composition may comprise an amphoteric surfactant, a zwitterionic surfactant, and mixtures thereof. Suitable zwitterionic surfactants typically contain both cationic and anionic groups in substantially equivalent proportions so as to be electrically neutral at the pH of use, and are well known in the art. Some common examples of zwitterionic surfactants are described in U.S. Pat. Nos. 2,082,275, 2,702,279 and 2,255,082.

Suitable zwitteronic surfactants include betaines such alkyl betaines, alkylamidobetaine, amidazoliniumbetaine, sulfobetaine (INCI Sultaines) as well as the phosphobetaine.

Suitable betaines are the alkyl betaines of the formula (Va), the alkyl amido betaine of the formula (Vb), the sulfo betaines of the formula (Vc) and the amido sulfobetaine of the formula (Vd);

R1-N+(CH3)2-CH2COO— (Va)

R1-CO—NH(CH2)3-N+(CH3)2-CH2COO— (Vb)

R1-N+(CH3)2-CH2CH(OH)CH2SO3- (Vc)

R1-CO—NH—(CH2)3-N+(CH3)2-CH2CH(OH)CH2SO3- (Vd)

in which R1 is a saturated or unsaturated C₆-C₂₂ alkyl residue, preferably C₈-C₁₈ alkyl residue. Particularly preferred are betaines of the formula Va such as for example N-alkyl-N-dimethyl betaine like the one sold under the trade name of Empigen® BB by Huntsman.

If the composition comprises a zwitterionic surfactant, it is preferably a betaine of the formula Va such as for example N-alkyl-N-dimethyl betaine like the one sold under the trade name of Empigen BB by Huntsman. It has been found these betaines greatly increase the antibacterial efficacy of the pyridinium esters.

Amphoteric surfactants can be either cationic or anionic depending upon the pH of the composition. Suitable amphoteric surfactants include the products sold under the trade name Miranol by Solvay-Novecare such as, for example, sodium lauroamphoacetate (Miranol Ultra L-32E), sodium stearoampho acetate (Miranol DM), disodium cocoamphodiacetate (Miranol C2m Conc NP), disodium lauroamphodiacetate (Miranol BM Conc), disodium capryloampho dipropionate (Miranol JBS), sodium mixed C₈ amphocarboxylate (Miranol JEM Conc), and sodium capryloampho hydroxypropyl sulfonate (Miranol JS). Other non-limiting examples of suitable amphoteric surfactants are disodium capryloamphodiacetate (Mackam 2CY 75-Solvay Novecare), octyliminodipropionate (Ampholak YJH40-Akzo Nobel), sodium lauriminodipropionate (Mirataine H2C-HA-Solvay Novecare), and sodium lauroamphohydroxypropylsulfonate (Mackam LS-Solvay Novecare). Amphoteric surfactants might not impact negatively the antimicrobial efficacy of the pyridinium esters.

Other suitable additional surfactants can be found in McCutcheon's Detergents and Emulsifiers, North American Ed. 1980.

### Cationic Surfactant

The compositions disclosed herein may comprise a cationic surfactant. Non-limiting examples of cationic surfactants include: the quaternary ammonium surfactants, which can have up to 26 carbon atoms and may include alkoxylate quaternary ammonium (AQA) surfactants, dimethyl hydroxyethyl quaternary ammonium, and/or dimethyl hydroxyethyl lauryl ammonium chloride; polyamine cationic surfactants; cationic ester surfactants; amino surfactants, e.g., amido propyldimethyl amine (APA); and mixtures thereof.

Suitable cationic surfactants also may include alkyl pyridinium compounds, alkyl quaternary ammonium compounds, alkyl quaternary phosphonium compounds, alkyl ternary sulphonium compounds, and mixtures thereof.

Suitable cationic detersive surfactants are quaternary ammonium compounds having the general formula:

(R)(R₁)(R₂)(R₃)N⁺X⁻

wherein, R is a linear or branched, substituted or unsubstituted C₆₋₁₈ alkyl or alkenyl moiety, R₁ and R₂ are independently selected from methyl or ethyl moieties, R₃ is independently selected from a linear or branched, substituted or unsubstituted C₆₋₁₈ alkyl or alkenyl moiety or a hydroxyl, hydroxymethyl or a hydroxyethyl moiety, X is an anion which provides charge neutrality, suitable anions include: halides, for example chloride; sulphate; and sulphonate. Suitable cationic surfactants are mono-C₆₋₁₈ alkyl mono-hydroxyethyl di-methyl quaternary ammonium chlorides. Highly suitable cationic surfactants are di-C₈₋₁₀ alkyl di-methyl quaternary ammonium chloride, mono-C₁₆ alkyl tri-methyl quaternary ammonium chloride, di-C₁₀₋₁₂ alkyl di-methyl quaternary ammonium chloride and mono-C₁₀ alkyl mono-hydroxyethyl di-methyl quaternary ammonium chloride.

### Anionic Surfactants

The composition may comprise anionic surfactants but preferably, the composition is free of anionic surfactants. By “free of anionic surfactant” is herein meant that the composition comprises less than 0.05% by weight of the composition of anionic surfactant.

Particularly preferred surfactants for use herein include nonionic surfactants, in particular branched alcohol alkoxylates, more in particular 2-ethyl hexan-1-ol ethoxylated to a degree of from 4 to 6, and propoxylated to a degree of from 4 to 6, more preferably, the alcohol is first propoxylated and then ethoxylated, and 2-alkyl-1-alkanols such as alkoxylated C₁₀ guerbet alcohols with 1 to 14, preferably 2 to 8, more preferably 3 to 6 ethoxylate or ethoxylate-propoxylate units. Other particularly preferred non-ionic surfactants include linear alcohol alkoxylated nonionic surfactants with C₈, C₁₀, C₁₂, C₁₄ mixtures of C₈ to C₁₀, mixtures of C₁₀ to C₁₂, mixtures of C₁₂ to C₁₄, mixtures of C₉ to C₁₁ linear alkyl chain and 10 or less ethoxylate units, preferably 3 to 9 ethoxylate units. Most preferably the alkoxylated nonionic surfactant is selected from the group consisting of: C₁₂-C₁₄ alcohol EO9 (Surfonic L 24-9), 2-propylheptyl EO8 (Lutensol XL89-BASF); 2-propylheptyl EO5 (Lutensol XL50-BASF); C₁₀ alcohol EO5 (Lutensol ON 50-BASF); C₁₀ alcohol EO7 (Lutensol ON 70-BASF); C₈-C₁₀ alcohol EO5 (Novel 810 FD5 Sasol); C₁₀ alcohol EO4 (Novel 10-4 Sasol); and 2-ethyl-hexanol PO5EO6 (Ecosurf EH6-Dow).

Other particularly preferred surfactants for use here in include linear amine oxide surfactants, in particular C₈-C₁₂ dimethyl amine oxide, more in particular C₁₀ dimethyl amine oxide; alkyldimethylbetaine surfactants, more in particular N,N-Dimethyl-N-dodecylglycine betaine (Empigen BB-Huntsman); alkyl glucamide surfactants such as N-alkyl-N-acylglucamide preferably N-decanoyl-N-methylglucamine, and the alkyl glucamide surfactants sold under the name of GlucoPure®, GlucoTain®, and GlucoWet® by Clariant; alkylpolyglucoside surfactants, more in particular C₈ to C₁₂ alkyl polyglucosides, more preferably Cs to C₁₀ alkyl polyglucosides such as for example Triton CG50 (Dow)

These surfactants improve the antimicrobial activity of the pyridinium esters.

### Non-aqueous Solvent

The antimicrobial system of the composition of the present disclosure comprises a non-aqueous solvent, the solvent comprises carbon, hydrogen and oxygen atoms. Preferred solvents for use herein include glycol ethers, diols and mixtures thereof. Suitable diols to use herein include 1,2-hexane diol, 1,2-octanediol and 1,4 butanediol. Especially preferred for use herein is 1,4-butanediol. The composition of the present disclosure preferably comprises a glycol ether of Formula VI:

Formula VI=R1O(R2O)nR3

wherein R1 is a linear or branched C4, C5 or C6 alkyl, a substituted or unsubstituted phenyl, preferably n-butyl. Benzyl is one of the substituted phenyls for use herein.
R2 is ethyl or isopropyl, preferably isopropyl
R3 is hydrogen or methyl, preferably hydrogen
n is 1, 2 or 3, preferably 1 or 2

Suitable glycol ethers according to Formula 1 include ethyleneglycol n-butyl ether, diethyleneglycol n-butyl ether, tricthyleneglycol n-butyl ether, propyleneglycol n-butyl ether, dipropyleneglycol n-butyl ether, tripropyleneglycol n-butyl ether, ethyleneglycol n-pentyl ether, diethyleneglycol n-pentyl ether, triethyleneglycol n-pentyl ether, propyleneglycol n-pentyl ether, dipropyleneglycol n-pentyl ether, tripropyleneglycol n-pentyl ether, ethyleneglycol n-hexyl ether, diethyleneglycol n-hexyl ether, triethyleneglycol n-hexyl ether, propyleneglycol n-hexyl ether, dipropyleneglycol n-hexyl ether, tripropyleneglycol n-hexyl ether, ethyleneglycol phenyl ether, diethyleneglycol phenyl ether, tricthyleneglycol phenyl ether, propyleneglycol phenyl ether, dipropyleneglycol phenyl ether, tripropyleneglycol phenyl ether, ethyleneglycol benzyl ether, diethyleneglycol benzyl ether, tricthyleneglycol benzyl ether, propyleneglycol benzyl ether, dipropyleneglycol benzyl ether, tripropyleneglycol benzyl ether, ethyleneglycol isobutyl ether, diethyleneglycol isobutyl ether, tricthyleneglycol isobutyl ether, propyleneglycol isobutyl ether, dipropyleneglycol isobutyl ether, tripropyleneglycol isobutyl ether, ethyleneglycol isopentyl ether, diethyleneglycol isopentyl ether, tricthyleneglycol isopentyl ether, propyleneglycol isopentyl ether, dipropyleneglycol isopentyl ether, tripropyleneglycol isopentyl ether, ethyleneglycol isohexyl ether, diethyleneglycol isohexyl ether, triethyleneglycol isohexyl ether, propyleneglycol isohexyl ether, dipropyleneglycol isohexyl ether, tripropyleneglycol isohexyl ether, ethyleneglycol n-butyl methyl ether, diethyleneglycol n-butyl methyl ether tricthyleneglycol n-butyl methyl ether. propyleneglycol n-butyl methyl ether, dipropyleneglycol n-butyl methyl ether, tripropyleneglycol n-butyl methyl ether, ethyleneglycol n-pentyl methyl ether, diethyleneglycol n-pentyl methyl ether. tricthyleneglycol n-pentyl methyl ether, propyleneglycol n-pentyl methyl ether, dipropyleneglycol n-pentyl methyl ether, tripropyleneglycol n-pentyl methyl ether, ethyleneglycol n-hexyl methyl ether, diethyleneglycol n-hexyl methyl ether, tricthyleneglycol n-hexyl methyl ether, propyleneglycol n-hexyl methyl ether, dipropyleneglycol n-hexyl methyl ether, tripropyleneglycol n-hexyl methyl ether, ethyleneglycol phenyl methyl ether, diethyleneglycol phenyl methyl ether, tricthyleneglycol phenyl methyl ether, propyleneglycol phenyl methyl ether, dipropyleneglycol phenyl methyl ether, tripropyleneglycol phenyl methyl ether, ethyleneglycol benzyl methyl ether, diethyleneglycol benzyl methyl ether, tricthyleneglycol benzyl methyl ether, propyleneglycol benzyl methyl ether, dipropyleneglycol benzyl methyl ether, tripropyleneglycol benzyl methyl ether, ethyleneglycol isobutyl methyl ether, diethyleneglycol isobutyl methyl ether. tricthyleneglycol isobutyl methyl ether, propyleneglycol isobutyl methyl ether, dipropyleneglycol isobutyl methyl ether, tripropyleneglycol isobutyl methyl ether, ethyleneglycol isopentyl methyl ether, diethyleneglycol isopentyl methyl ether, tricthyleneglycol isopentyl methyl ether, propyleneglycol isopentyl methyl ether, dipropyleneglycol isopentyl methyl ether. tripropyleneglycol isopentyl methyl ether, ethyleneglycol isohexyl methyl ether, diethyleneglycol isohexyl methyl ether, triethyleneglycol isohexyl methyl ether, propyleneglycol isohexyl methyl ether, dipropyleneglycol isohexyl methyl ether, tripropyleneglycol isohexyl methyl ether, and mixtures thereof.

Preferred glycol ether solvents according to Formula VI are ethyleneglycol n-butyl ether. diethyleneglycol n-butyl ether, triethyleneglycol n-butyl ether, propyleneglycol n-butyl ether, dipropyleneglycol n-butyl ether, tripropyleneglycol n-butyl ether, and mixtures thereof.

The most preferred glycol ether for use herein are selected from is the group consisting of of diethylene glycol butyl ether, di(propylene glycol) methyl ether and mixtures thereof.

The composition of the present disclosure preferably comprises from about 0.1% to about 10%, more preferably from about 0.2 to about 6% by weight of the composition of the glycol ether, more preferably from about 0.2 to about 3% by weight of the composition of diethylene glycol butyl ether, di(propylene glycol) methyl ether and mixtures thereof.

### Optional Ingredients

### Chelating Agent

The antimicrobial composition can comprise a chelating agent. Suitable chelating agents, in combination with the surfactant system, improve the shine benefit. Chelating agent can be incorporated into the compositions in amounts ranging from 0.02% to 5.0%, preferably from 0.1% to 3.0%, more preferably from 0.2% to 2.0% and most preferably from 0.2% to 0.4% by weight of the composition.

Suitable phosphonate chelating agents include ethylene diamine tetra methylene phosphonates, and diethylene triamine penta methylene phosphonates (DTPMP), and can be present either in their acid form or as salts.

A preferred biodegradable chelating agent for use herein is ethylene diamine N,N′-disuccinic acid, or alkali metal, or alkaline earth, ammonium or substitutes ammonium salts thereof or mixtures thereof. A more preferred biodegradable chelating agent is L-glutamic acid N,N-diacetic acid (GLDA) commercially available under tradename Dissolvine 47S from Akzo Nobel.

Suitable amino carboxylates include ethylene diamine tetra acetates, diethylene triamine pentaacetates, diethylene triamine pentaacetate (DTPA), N-hydroxyethylethylenediamine triacetates, nitrilotriacetates, ethylenediamine tetrapropionates, triethylenetetraaminchexa-acetates, ethanoldiglycines, and methyl glycine diacetic acid (MGDA), both in their acid form, or in their alkali metal, ammonium, and substituted ammonium salt forms. Particularly suitable amino carboxylate to be used herein is propylene diamine tetracetic acid (PDTA) which is, for instance, commercially available from BASF under the trade name Trilon FS® and methyl glycine di-acetic acid (MGDA). Most preferred aminocarboxylate used herein is diethylene triamine pentaacetate (DTPA) from BASF. Further carboxylate chelating agents for use herein include salicylic acid, aspartic acid, glutamic acid, glycine, malonic acid or mixtures thereof.

Suitable polycarboxylates include itaconic acid and sodium polyitaconate which is, for instance, commercially available from Itaconix under the trade name of Itaconix® DSP 2K™, and Itaconix® CHT121™.

### Polymers

The antimicrobial composition may comprise an additional polymer. It has been found that the presence of a specific polymer as described herein, when present, allows further improving the grease removal performance of the composition due to the specific sudsing/foaming characteristics they provide to the composition. Suitable polymers for use herein are disclosed in EP2272942 and EP2025743.

The polymer can be selected from the group consisting of: a vinylpyrrolidone homopolymer (PVP); a polyethyleneglycol dimethylether (DM-PEG); a vinylpyrrolidone/dialkylaminoalkyl acrylate or methacrylate copolymers; a polystyrenesulphonate polymer (PSS); a poly vinyl pyridine-N-oxide (PVNO); a polyvinylpyrrolidone/vinylimidazole copolymer (PVP-VI); a polyvinylpyrrolidone/polyacrylic acid copolymer (PVP-AA); a polyvinylpyrrolidone/vinylacetate copolymer (PVP-VA); a polyacrylic polymer or polyacrylicmaleic copolymer; and a polyacrylic or polyacrylic maleic phosphono end group copolymer; and mixtures thereof.

Typically, the antimicrobial hard surface cleaning composition may comprise from 0.005% to 5.0%, preferably from 0.10% to 4.0%, more preferably from 0.1% to 3.0% and most preferably from 0.20% to 1.0% by weight of the composition of said polymer.

### Thickener

The antimicrobial composition of the present disclosure can further comprise a thickener. Suitable thickeners herein include polyacrylate based polymers, preferably hydrophobically modified polyacrylate polymers; amide polymers; hydroxyl ethyl cellulose, preferably hydrophobically modified hydroxyl ethyl cellulose, xanthan gum, hydrogenated castor oil (HCO) and mixtures thereof.

### Other Optional Ingredients

The composition of the present disclosure may comprise a variety of other optional ingredients depending on the technical benefit aimed for and the surface treated. Suitable optional ingredients for use herein include perfume, builders, other polymers, buffers, hydrotropes, colorants, stabilisers, radical scavengers, abrasives, soil suspenders, brighteners, anti-dusting agents, dispersants, dye transfer inhibitors, pigments, silicones and/or dyes.

A preferred composition according to the present disclosure comprises
a) from 0.01% to 5% by weight of the composition of a non-ionic surfactant selected from the group consisting of alkyl polyglucoside, alkoxylated alcohol and mixtures thereof;
b) from 0.01% to 10% by weight of the composition of a solvent is selected from the group consisting of diethylene glycol butyl ether, di(propylene glycol) methyl ether and mixtures thereof;
c) from 0.001% to 1% by weight of the composition of a pyridinium ester according to Formula I, preferably wherein one of R2 and R3 is H and the other is Formula III, all R4 are H, R1 and R5 are independently selected from a branched or unbranched C₆-C₈ saturated hydrocarbyl groups; and
d) at least 50% by weight of the composition of water.
   A preferred composition according to the present disclosure comprises
a) from 0.01% to 5% by weight of the composition of a non-ionic surfactant comprising a fatty alcohol preferably an alkoxylated alcohol comprising from 6 to 16 carbon atoms and from 2 to 12 alkoxy groups, preferably from 4 to 10 ethoxy groups or a branched ethoxylated propoxylated alcohol;
b) from 0. 01% to 10% by weight of the composition of a solvent is selected from the group consisting of glycol ethers, diols and mixtures thereof;
c) from 0.001 to 1% by weight of the composition of a pyridinium ester according to Formula I, preferably wherein one of R2 and R3 is H and the other is Formula III, all R4 are H, R1 and R5 are independently selected from a branched or unbranched C₆-C₈ saturated hydrocarbyl groups;
d) from 85% to 98% by weight of the composition of water.
   A preferred composition according to the present disclosure comprises
a) from 0.01% to 5% by weight of the composition of a non-ionic surfactant selected from the group consisting of alkyl polyglucoside, alkoxylated alcohols and mixtures thereof;
b) from 0.01% to 10% by weight of the composition of a solvent is selected from the group consisting of diethylene glycol butyl ether, di(propylene glycol) methyl ether and mixtures thereof;
c) from 0.001 to 1% by weight of the composition of a pyridinium ester according to Formula I, preferably wherein one of R2 and R3 is H and the other is Formula III, all R4 are H, R1 and R5 are independently selected from a branched or unbranched C₆-C₈ saturated hydrocarbyl groups;
d) from 85% to 98% by weight of the composition of water.

### Wipe

The present disclosure also relates to an article treated with the composition of the present disclosure. The article is preferably a wipe. Suitable wipes can be fibrous. Suitable fibrous wipes can comprise polymeric fibres, cellulose fibres, and combinations thereof. Suitable cellulose-based wipes include kitchen wipes, and the like. Suitable polymeric fibres include polyethylene, polyester, and the like. Polymeric fibres can be spun-bonded to form the wipe. Methods for preparing thermally bonded fibrous materials are described in U.S. application Ser. No. 08/479,096 (see especially pages 16-20) and U.S. Pat. No. 5,549,589 (see especially Columns 9 to 10). Suitable pads include foams and the like, such as HIPE-derived hydrophilic, polymeric foam. Such foams and methods for their preparation are described in U.S. Pat. No. 5,550,167; and U.S. patent application Ser. No. 08/370,695.

The load factor is defined as the weight ratio of antimicrobial solution to nonwoven substrate is preferably from about 3× to about 10×. Preferably, the load factor is between 4× and 8×, or from 4.5× to 7.5×, or from 5× to 7×. It is found that higher load factors for the pre-moistened wipes of the present disclosure are preferable since they help increase product mileage.

### Method of Cleaning a Surface

The cleaning composition of the present disclosure is particularly suited for cleaning surfaces selected from the group consisting of: ceramic, enamel, stainless steel, Inox®, Formica®, vinyl, no-wax vinyl, linoleum, melamine, glass, plastics and plastified wood, and combinations thereof. In particular, the compositions are particularly suited for reducing the microbial population, while leaving surfaces clean, shiny and grease free.

The compositions described herein can be used neat or can be achieved by diluting with water a concentrated composition prior to applying to the surface. In preferred methods, the hard surface cleaning composition is applied neat, more preferably, the hard surface cleaning composition is sprayed onto the hard surface.

The composition can be applied by any suitable means, including using a mop, sponge, cloth, paper towel, or other suitable implement.

The surface may be rinsed, preferably with clean water, in an optional further step, and also as a further step, wiped, such as with a cloth or a paper towel.

In another preferred embodiment of the present disclosure said method of cleaning a surface includes the steps of applying, preferably spraying, said liquid composition onto said hard surface, leaving said liquid composition to act onto said surface for a period of time with or without applying mechanical action, and optionally removing said liquid composition, preferably removing said liquid composition by rinsing said hard surface with water and/or wiping said hard surface with an appropriate implement, e.g., a sponge, a paper or cloth towel and the like. Such compositions are often referred to as “ready-to-use” compositions. In preferred methods, the surface is not rinsed after application of the antimicrobial composition.

It is believed that antimicrobial compositions comprising specific surfactants, non-aqueous solvents and the pyridinium ester of Formula I deliver very good antimicrobial efficacy at very low levels of antimicrobial agent. The antimicrobial hard surface cleaning composition of the present disclosure exhibits improved antimicrobial efficacy, good grease cleaning and/or streak-free shine.

### Test Methods

### pH Measurement

The pH is measured on the neat composition, at 25° C., using a pH meter with compatible gel-filled pH probe (such as Sartarius PT-10P meter with Toledo probe part number 52 000 100), calibrated according to the instruction manual.

### Surface Antimicrobial Kill Testing Method

Inoculum is prepared by streaking microorganism onto a Tryptic Soy Agar (TSA) plate and incubating for 24 h at 33° C. Plated bacteria is resuspended in saline until the transmittance percentage falls within the accepted range for ˜10⁸ cfu/mL. For K. pneumonia (Kp) and P. aeruginosa (Pa), this is 31.00-33.00% T. For S. aureus (Sa) this is 23.00-25.00% T. A 10× concentration is made by spinning down the prepared inoculum, removing the supernatant saline from the bacterial pellet, then resuspending the pellet in 1/10th of the original volume of saline. The final inoculum is prepared with 5% of “soil” in the form of Fetal Bovine Serum (FBS).

A 18×18 mm sterile glass coverslip is placed into an appropriate container (e.g. petri dish). 20 μL of prepared inoculum is added to the glass coverslip. Inoculum is then air-dried for 30 minutes in a 36° C. incubator. After drying, 40 μL of test sample is added on top of the dried inoculum. This is incubated at room temperature for the desired contact time. 5 mL of neutralizing media (Modified Letheen Broth+Tween+Lecithin (MLBTL)) is added to the coverslip and shaken on an orbital shaker for 5 minutes to ensure proper neutralization and extraction. Viability is then assessed of the neutralized and extracted sample by traditional/alternative microbiological techniques.

### Normalization of Antimicrobial Activity Method

The normalized antimicrobial activity reported in the performance examples below was generated from detection time data obtained as described in the Surface Antimicrobial Kill Test Method. To account for day-to-day variability an internal standard was tested with each series. The internal standard was a composition of 0.25 wt % Triclosan, Sigma Aldrich (St. Louis, MO, USA; CAS #3380-34-5, 97.0-103.0% (active substance, GC)), 50 wt % DMSO, and 49.75 wt % H₂O, with a final solution pH of 7.7. Normalized activity is reported following the equation 1 below.

Normalized Activity=(αΔDT-βΔDT)/(γΔDT-βΔDT) equation 1.

where, αΔDT is the average detection time of the antimicrobial compound, and γΔDT is the average detection time of the reference triclosan internal standard, and βΔDT is the average detection time of the background unperturbed bacteria.

### Formulation Method for Surface Kill Testing

Pyridinium esters of the present disclosure were tested as aqueous solutions in the Surface Antimicrobial Kill Testing Method above. To prepare test solutions pyridinium esters were first dissolved in the appropriate solvent. Second the selected surfactant is added to the mixture. After vortex (1 min, Vortex-2 Genie, vortex speed 8) mixing, the samples were then diluted with ½ portion of reverse osmosis water (RO water, Millipore Direct-Q™, pH 6). The aqueous solution was then vortexed (1 min, Vortex-2 Genie, vortex speed 8), to which the second ½ portion of water is added. The pH of the solution was then adjusted to the target pH using either 1N HCl, Glacial Acetic Acid, or 1N NaOH. The final solution was then vortexed (1 min, Vortex-2 Genic, vortex speed 8). An example batch sheet is provided below in Table 1.

Surfactant description and supplier: Tween® 80 (Sigma Aldrich, St. Louis, MO, USA), Plantaren® 2000 N UP (BASF, Florham Park, NJ, USA), EcoSurf™ EH6 (Sigma Aldrich, St. Louis, MO, USA), EcoSurf™ EH9 (Sigma Aldrich, St. Louis, MO, USA), Surfonic® L24-9 (Huntsman Holland BV, Botlek-Rotterdam, Netherlands), Miranol® Ultra L-32 (Solvay S.A., Brussels, Belgium), Cetyltrimethylammonium chloride (CTAC, 25 wt % in H2O, Sigma Aldrich, St. Louis, MO, USA), Sodium Lauryl Sulfate (SLS, 29 wt % in H2O, Stepan, Northfield, IL, USA), Tergitol™ TMN-6 (Sigma Aldrich, St. Louis, MO, USA), C₁₀ dimethyl amine oxide is commercially available under the trade name Genaminox® K-10 from Clariant.

Solvents and supplier: 1,4-Butanediol (99%, Sigma Aldrich, St. Louis, MO, USA), Ethylene Glycol (99%, Sigma Aldrich, St. Louis, MO, USA), Diethylene glycol mono-n-butyl ether (99%, DEGBE, Thermo Fisher Scientific, Ward Hill, MA, USA), Di(propylene glycol)methyl ether, mixture of isomers (99%, DPGME, Sigma Aldrich, St. Louis, MO, USA), Dimethyl sulfoxide (99%, DMSO, igma Aldrich, St. Louis, MO, USA), 1,2-Hexanediol (98%, Sigma Aldrich. St. Louis, MO. USA). Hexylene Glycol (99%. Sigma Aldrich. St. Louis, MO. USA). 2-Phenoxyethanol (99%. Sigma Aldrich. St. Louis, MO. USA). Di(ethylene glycol) hexyl ether (95%. Sigma Aldrich, St. Louis, MO. USA).

TABLE 1
Illustrative formulation sheet for hexyl 3-(alkylpyridinium)acrylate
halide solutions for surface antimicrobial kill testing.

| Ingredients | Quantity (g) |
|---|---|
| Hexyl 3-(alkylpyridinium)acrylate halide | 0.05 |
| EcoSurf EH-9 | 0.1 |
| DEGBE | 0.5 |
| Reverse Osmosis Water | 9.35 |
| Total Batch Size | 10.0 |

TABLE 2
Illustrative formulation sheet for hexyl 3-(alkylpyridinium)acrylate
halide solutions for surface antimicrobial kill testing.

| Ingredients | Quantity (g) |
|---|---|
| Hexyl 3-(alkylpyridinium)acrylate halide | 0.05 |
| EcoSurf EH-9 | 0.1 |
| 1,2-Hexanediol | 0.5 |
| Reverse Osmosis Water | 9.35 |
| Total Batch Size | 10.0 |

TABLE 3
Illustrative formulation sheet for hexyl 3-(alkylpyridinium)acrylate
halide solutions for surface antimicrobial kill testing.

| Ingredients | Quantity (g) |
|---|---|
| Hexyl 3-(alkylpyridinium)acrylate halide | 0.05 |
| EcoSurf EH-9 | 0.1 |
| Hexylene Glycol | 0.5 |
| Reverse Osmosis Water | 9.35 |
| Total Batch Size | 10.0 |

TABLE 4
Illustrative formulation sheet for hexyl 3-(alkylpyridinium)acrylate
halide solutions for surface antimicrobial kill testing.

| Ingredients | Quantity (g) |
|---|---|
| Hexyl 3-(alkylpyridinium)acrylate halide | 0.05 |
| Surfonic L 24-9 | 0.1 |
| Ethylene Glycol | 0.5 |
| Reverse Osmosis Water | 9.35 |
| Total Batch Size | 10.0 |

TABLE 5
Illustrative formulation sheet for hexyl 3-(alkylpyridinium)acrylate
halide solutions for surface antimicrobial kill testing.

| Ingredients | Quantity (g) |
|---|---|
| Hexyl 3-(alkylpyridinium)acrylate halide | 0.05 |
| Tergitol TMN-6 | 0.1 |
| Di(ethylene glycol) hexyl ether | 0.5 |
| Reverse Osmosis Water | 9.35 |
| Total Batch Size | 10.0 |

TABLE 6
Illustrative formulation sheet for hexyl 3-(alkylpyridinium)acrylate
halide solutions for surface antimicrobial kill testing.

| Ingredients | Quantity (g) |
|---|---|
| Hexyl 3-(alkylpyridinium)acrylate halide | 0.05 |
| EcoSurf EH-6 | 0.1 |
| 2-Phenoxyethanol | 0.5 |
| Reverse Osmosis Water | 9.35 |
| Total Batch Size | 10.0 |

TABLE 7
Illustrative formulation sheet for hexyl 3-(alkylpyridinium)acrylate
halide solutions for surface antimicrobial kill testing.

| Ingredients | Quantity (g) |
|---|---|
| Hexyl 3-(alkylpyridinium)acrylate halide | 0.05 |
| EcoSurf EH-9 | 0.1 |
| 2-Phenoxyethanol | 0.1 |
| DPGME | 0.4 |
| Reverse Osmosis Water | 9.35 |
| Total Batch Size | 10.0 |

TABLE 8
Illustrative formulation sheet for hexyl 3-(alkylpyridinium)acrylate
halide solutions for surface antimicrobial kill testing.

| Ingredients | Quantity (g) |
|---|---|
| Hexyl 3-(alkylpyridinium)acrylate halide | 0.05 |
| Genaminox | 0.1 |
| DEGBE | 0.5 |
| Reverse Osmosis Water | 9.35 |
| Total Batch Size | 10.0 |

TABLE 9
Illustrative formulation sheet for hexyl 3-(alkylpyridinium)acrylate
halide solutions for surface antimicrobial kill testing.

| Ingredients | Quantity (g) |
|---|---|
| Hexyl 3-(alkylpyridinium)acrylate halide | 0.05 |
| Didecyldimethylammonium | 0.5 |
| chloride | |
| DEGBE | 0.5 |
| Reverse Osmosis Water | 9.4 |
| Total Batch Size | 10.0 |

TABLE 10
Illustrative formulation sheet for hexyl 3-(alkylpyridinium)acrylate
halide solutions for surface antimicrobial kill testing.

| Ingredients | Quantity (g) |
|---|---|
| Hexyl 3-(alkylpyridinium)acrylate halide | 0.05 |
| Surfonic L 24-9 | 0.05 |
| Tween 80 | 0.05 |
| DEGBE | 0.5 |
| Reverse Osmosis Water | 9.35 |
| Total Batch Size | 10.0 |

TABLE 11
Illustrative formulation sheet for hexyl 3-(alkylpyridinium)acrylate
halide solutions for surface antimicrobial kill testing.

| Ingredients | Quantity (g) |
|---|---|
| Hexyl 3-(alkylpyridinium)acrylate halide | 0.025 |
| EcoSurf EH-9 | 0.05 |
| DEGBE | 0.25 |
| Reverse Osmosis Water | 9.675 |
| Total Batch Size | 10.0 |

TABLE 12
Illustrative formulation sheet for hexyl 3-(alkylpyridinium)acrylate
halide solutions for surface antimicrobial kill testing.

| Ingredients | Quantity (g) |
|---|---|
| Hexyl 3-(alkylpyridinium)acrylate halide | 0.05 |
| Sodium Lauryl Sulfate (SLS) | 0.05 |
| Tween 80 | 0.05 |
| DEGBE | 0.5 |
| Reverse Osmosis Water | 9.35 |
| Total Batch Size | 10.0 |

### Synthesis & Comparative Examples

### Methods of Preparing Pyridinium Esters

In the following synthesis examples, the materials are generally obtained/available from Sigma-Aldrich (St. Louis, MO, USA), except as indicated below. The carboxaldehydes are generally provided at >97% purity. The alkyl halides are generally provided at >98% or >99% purity. Monohexylmalonate is prepared according to known procedures (European Journal of Medicinal Chemistry 46 (2011) 773-777).

### General Method A: General Preparation of Hexyl 3-(pyridyl)acrylate Compounds

To prepare a hexyl 3-(pyridyl)acrylate, a round bottom flask is charged with 1 equiv. of a pyridine carboxaldehyde, 1.1 equiv. monohexylmalonate, 1 equiv. pyridine, and 1.6 equiv. β-alanine as catalysts. The flask is then diluted with acetonitrile and refluxed for 24 h using a dean-stark apparatus. The resulting mixture is acidified with HCl, diluted with H₂O and washed with EtOAc. The aqueous layer is neutralized with sodium bicarbonate followed by extraction of the product with EtOAc. The organic phase is dried over MgSO₄ and filtered. The solvent is removed to yield the hexyl 3-(pyridyl)acrylate.

### Synthetic Example 1

Synthetic Example 1, hexyl 3-(3-pyridyl)acrylate, was prepared as described in General Method A, but using 10.7 g of nicotinaldehyde, 20.7 g of hexyl malonate, 7.9 g of pyridine, and 14.3 g of β-alanine.

### Synthetic Example 2

Synthetic Example 2, hexyl 3-(4-pyridyl)acrylate, was prepared as described in General Method A, but using 3.21 g of 4-pyridylcarboxaldehyde, 6.21 g of hexyl malonate, 2.37 g of pyridine, and 4.28 g of β-alanine.

### General Method B: General Preparation of Hexyl 3-(methylpyridinium)acrylate Iodide Compounds

To prepare a hexyl 3-(methylpyridinium)acrylate iodide, a round bottom flask is charged with 1 equiv. of a hexyl 3-(pyridyl)acrylate and 2 equiv. methyl iodide. The flask is then diluted with acetonitrile and refluxed for 4 h. The pyridinium product is precipitated through addition of ether.

### Synthetic Example 3

Synthetic Example 3, hexyl 3-(3-methylpyridinium)acrylate iodide, was prepared as described in General Method B, but using 4 g of Synthetic Example 1 and 4.87 g of methyl iodide. The independent solid 3 appears stable for several months by 1H NMR.

### Synthetic Example 4

Synthetic Example 4, hexyl 3-(4-methylpyridinium)acrylate iodide, was prepared as described in General Method B, but using 4 g of Synthetic Example 2 and 4.87 g of methyl iodide. The independent solid 4 appears stable for several months by 1H NMR.

### General Method C: General Preparation of Hexyl 3-(alkylpyridinium)acrylate Bromide Compounds

To prepare a hexyl 3-(alkylpyridinium)acrylate bromide, a round bottom flask is charged with 1 equiv. of a hexyl 3-(pyridyl)acrylate and 1.5-2 equiv. of a bromoalkane. The flask is then diluted with acetonitrile and refluxed for 3 days. The pyridinium product is precipitated through addition of ether.

### Synthetic Example 5

Synthetic Example 5, hexyl 3-(3-hexylpyridinium)acrylate bromide, was prepared as described in General Method C, but using 2.3 g of Synthetic Example 1 and 3.0 g of bromohexane. The independent solid 5 appears stable for several months by 1H NMR.

### Synthetic Example 6

Synthetic Example 6, hexyl 3-(4-hexylpyridinium)acrylate bromide, was prepared as described in General Method C, but using 2.3 g of Synthetic Example 2 and 3.0 g of bromohexane. The independent solid 6 appears stable for several months by 1H NMR.

### Synthetic Example 7

Synthetic Example 7, hexyl 3-(3-octylpyridinium)acrylate bromide, was prepared as described in General Method C, but using 3.0 g of Synthetic Example 1 and 5.0 g of bromooctane. The independent solid 7 appears stable for several months by 1H NMR.

### Synthetic Example 8

Synthetic Example 8, hexyl 3-(4-octylpyridinium)acrylate bromide, was prepared as described in General Method C, but using 2.3 g of Synthetic Example 2 and 3.0 g of bromooctane. The independent solid 8 appears stable for several months by 1H NMR.

### Structures of the Synthetic Examples

TABLE 13
Structural representation of the Synthetic Examples 1-8.

| No. | Structure |
|---|---|
| 1 | 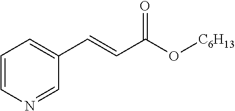 |
| 2 | 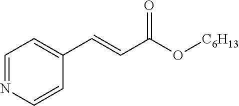 |
| 3 | 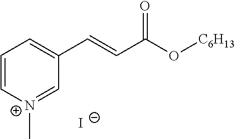 |
| 4 | 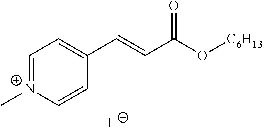 |
| 5 | 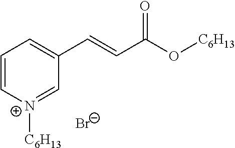 |
| 6 | 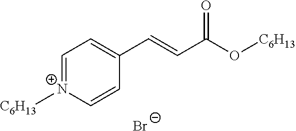 |
| 7 | 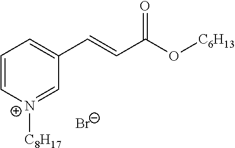 |
| 8 | 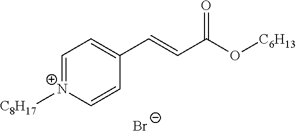 |

### Performance Examples

In Performance Examples 1-4 below, formulations comprising solvent, surfactant, and pyridinium esters according to the present disclosure (e.g., based on hexyl 3-(alkylpyridinium)acrylate halides), are evaluated via microbial surface kill tests according to the Surface Antimicrobial Kill Testing Method provided above. After treatment, microbial growth is referenced and reported to a triclosan sample and unaltered microbes according to the Normalization of Antimicrobial Activity provided above. Therefore, values greater than one would be more active above the internal triclosan control, with values less than one but greater than zero still having antimicrobial activity. The data below shows the benefits afforded by hexyl 3-10 (alkylpyridinium)acrylate halides.

### Performance Example 1. Antimicrobial Activity of Formulations of Synthetic Examples 1-8

TABLE 14
Antimicrobial Activity of Formulations of Synthetic Examples.

| Antimicrobial | Gram Positive Referenced |
|---|---|
| Compound (0.5 wt %)^{a} | Microbial Activity |
| Synthetic Example 1 | 0.11 |
| Synthetic Example 3 | 0.21 |
| Synthetic Example 4 | 1.2 |
| Synthetic Example 5 | >1.42 |
| Synthetic Example 6 | >1.39 |
| Synthetic Example 7 | >1.42 |
| Synthetic Example 8 | >1.33 |

^{a}Remaining wt % fraction composed of 3 wt % DEGBE, 1 wt % EcoSurf EH-9, 95.5 wt % reverse osmosis water at pH 7.

As shown in Table 14, alkylation of the pyridyl structure is necessary for antimicrobial activity against staphylococcus aureus. A strong preference is given towards C₆-C₈ carbon chain lengths. Location of the pyridinium in the para position relative to the acrylate provides higher antimicrobial efficacy with shorter chain lengths while the pyridinium in the meta position is more effective with longer chain lengths.

### Performance Example 2. Examination of Surfactant on Surface Antimicrobial Efficacy of Synthetic Example 5

TABLE 15
Examination of surfactant on surface antimicrobial
efficacy of Synthetic Example 5.

| | | | Gram Negative | Gram Positive |
|---|---|---|---|---|
| | | | Referenced | Referenced |
| | Surfactant | % Synthetic | Microbial | Microbial |
| Row | (1 wt %)^{a} | Example 5 | Activity | Activity |
| 1 | EH6 | — | 0.18 | 0.07 |
| 2 | L24-9 | — | −0.06 | 0.06 |
| 3 | Tween80 | — | −0.10 | 0.01 |
| 4 | Miranol Ultra | — | 0.36 | 0.10 |
| 5 | Plantaren 2000 | — | 0.33 | 0.23 |
| 6 | Crodasinic LS30 | — | 0.16 | 0.29 |
| 7 | SLS | — | 0.13 | 0.22 |
| 8 | EH6 | 0.25 | >2.58 | 1.09 |
| 9 | L24-9 | 0.25 | 1.34 | 0.54 |
| 10 | Tween80 | 0.25 | 1.37 | 0.57 |
| 11 | Miranol Ultra | 0.25 | 1.92 | 0.78 |
| 12 | Plantaren 2000 | 0.25 | >2.58 | >2.33 |
| 13 | Crodasinic LS30 | 0.25 | 0.41 | 0.42 |
| 14 | SLS | 0.25 | 0.08 | 0.20 |

^{a}Remaining wt % fraction composed of 3 wt % DEGBE, 95.75 wt % reverse osmosis water at pH 7.

As shown in table 15, formulations containing Tween® 80, Plantaren® 2000 N UP, EcoSurf™ EH6, EcoSurf™ EH9, Surfonic® L24-9, or Miranol® Ultra L-32 all show an improvement in microbial activity over the surfactant, solvent, chassis alone. Combining Synthetic Example 5 with APG's, alcohol ethoxylates, and amphoteric surfactants resulted in measurable microbial activity. A preference is noted for APG's, row 12, with strong performance in gram-negative and gram-positive bacteria. Within alcohol ethoxylate surfactants a preference is given for shorter chain alcohols. Compositions containing anionic surfactants illustrated with sodium lauryl sulfate and Crodasinic LS30 had reduced activity. Without wishing to be bound by theory we asses this reduction in activity to ion paring.

### Performance Example 3. Examination of pH on Surface Antimicrobial Efficacy of Synthetic Example 4

TABLE 16
Examination of pH on surface antimicrobial
efficacy of Synthetic Example 4.

| pH^{a} | Gram Positive Referenced Microbial Activity |
|---|---|
| 3 | >2.58 |
| 7 | >2.8 |
| 9 | 2.53 |

^{a}Formulations composed of 0.5 wt % Synthetic Example 5, 1 wt % EcoSurf EH9, 3 wt % DEGBE, 95.5 wt % reverse osmosis water. pH adjusted with either HCl or NaOH.

As shown in table 16, Synthetic Example 5 maintains antimicrobial efficacy in formulations of 10 pH ranging from at least 3-9.

### Performance Example 4. Examination of Concentration on Surface Antimicrobial Efficacy of Synthetic Example 5

TABLE 17
Examination of concentration on surface antimicrobial
efficacy of Synthetic Example 5.

| Synthetic Example 5 | Gram Positive Referenced |
|---|---|
| wt %^{a} | Microbial Activity |
| 0.5 | >2.87 |
| 0.25 | 2.02 |
| 0.125 | 0.93 |
| 0.063 | 0.55 |

^{a}Formulations composed of 1 wt % EcoSurf EH9, 3 wt % DEGBE, and remaining wt % fraction composed of water at pH 7.

As shown in table 17, Synthetic Example 5 maintains antimicrobial efficacy against staphylococcus aureus in concentrations down to 0.063 wt %.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

What is claimed is:

1. A pyridinium ester according to Formula I 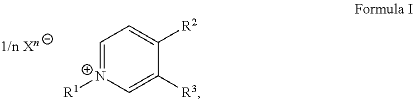
wherein 1/n Xⁿ⁻ is a counterion and n is 1 or 2;
wherein one of R² and R³ is H and the other is selected from the group consisting of Formula II, Formula III and Formula IV 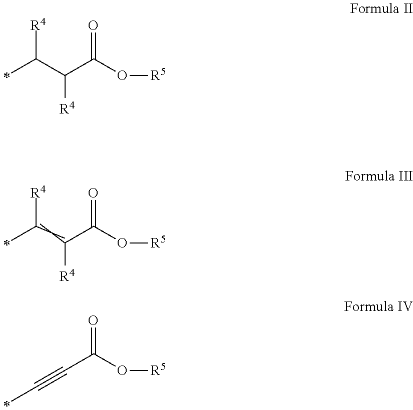
wherein * represents the point of attachment to the heteroaryl ring,
wherein Formula III is selected from the group consisting of a Z-configuration, an E-configuration, and mixtures thereof,
wherein each R⁴ is independently selected from hydrogen and a C₁-C₄ hydrocarbyl group
and
wherein R⁵ is a branched or unbranched, saturated or unsaturated, C₄-C₁₂ hydrocarbyl group,
wherein when R² is H, R¹ is a branched or unbranched, saturated or unsaturated, C₄-C₁₂ hydrocarbyl group;
when R³ is H, R¹ is a branched or unbranched, saturated or unsaturated, C₁-C₁₂ hydrocarbyl group.

2. The pyridinium ester according to claim 1, wherein one of R² and R³ is H and the other is Formula III.

3. The pyridinium ester according to claim 1, wherein the pyridinium ester is selected from the group consisting of: 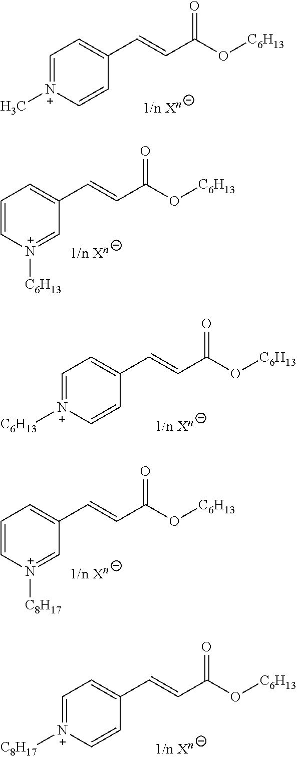
and a mixture thereof.

4. An antimicrobial composition comprising an antimicrobial system, the antimicrobial system comprising:
a. a surfactant;
b. a solvent; and
c. the pyridinium ester according to claim 1.

5. The antimicrobial composition according to claim 4, wherein the antimicrobial system comprises:
a. from about 0.01 to about 10% by weight of the composition of a surfactant selected from the group consisting of non-ionic surfactant, cationic surfactant, amphoteric surfactant and a mixture thereof;
b. from about 0.01 to about 10% by weight of the composition of a non-aqueous solvent; and
c. the pyridinium ester according to claim 1.

6. The antimicrobial composition according to claim 4, wherein the composition comprises a non-ionic surfactant or amphoteric surfactant selected from the group consisting of alkyl polyglucoside, alkoxylated alcohol, alkyl betaine, alkyl glucamine, alkyl glucamide, alkyl amine oxide and mixtures thereof, preferably the composition comprises a non-ionic surfactant selected from the group consisting of alkyl polyglucoside, alkoxylated alcohol and mixtures thereof.

7. The antimicrobial composition according to claim 6, wherein the non-ionic surfactant comprises an alkyl polyglucoside.

8. The antimicrobial composition according to claim 4, wherein the non-aqueous solvent is selected from the group consisting of glycol ethers, diols and mixtures thereof, preferably the non-aqueous solvent is selected from the group consisting of diethylene glycol butyl ether, di(propylene glycol) methyl ether and mixtures thereof.

9. The antimicrobial composition according to claim 4, further comprising:
a. from about 0.01 to about 10% by weight of the composition of a of non-ionic surfactant selected from the group consisting of alkyl polyglucoside, alkoxylated alcohol and mixtures thereof; and
b. from about 0.01 to about 10% by weight of the composition of a non-aqueous solvent selected from the group consisting of diethylene glycol butyl ether, di(propylene glycol) methyl ether and mixtures thereof.

10. The antimicrobial composition according to claim 4, wherein the composition comprises at least 80% by weight of the composition of water.

11. The antimicrobial composition according to claim 4, wherein the composition is in the form of a clear liquid.

12. The antimicrobial composition according to claim 4, wherein the composition is in the form of a ready-to-use spray.

13. The antimicrobial composition according to claim 4, wherein the composition comprises an additional benefit agent selected from the group consisting of a perfume raw material including aldehydes and/or ketones, an additional antimicrobial agent, a pesticide, an insect repellant, an anti-fungal agent, an antiviral agent, a herbicidal agent, a hueing dye, an antioxidant, a non-perfume organoleptic, a polymer, an abrasive, a stabilizer, a bitterant, a microcapsule or a combination thereof.

14. An article treated with an antimicrobial composition according to claim 4, wherein the article is in the form of a disposable or partially reusable substrate comprising one or more nonwoven layers and preferably the substrate has a load factor of from about 3 times to about 10 times of composition per gram of nonwoven substrate.

15. A method of sanitizing an inanimate surface comprising the step of contacting the surface with an antimicrobial composition or article according to claim 4.

16. The antimicrobial composition according to claim 1, wherein:
Formula III is an E-configuration,
wherein R⁵ is a branched or unbranched C₆-C₈ saturated hydrocarbyl group.

17. The antimicrobial composition according to claim 1, wherein Formula III is

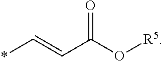

18. The antimicrobial composition according to claim 17, wherein R⁵ is selected from a branched or unbranched C₆-C₈ saturated hydrocarbyl group.

19. An antimicrobial composition comprising an antimicrobial system, the antimicrobial system comprising:
a. a surfactant;
b. a solvent; and
c. the pyridinium ester according to claim 3.

20. The antimicrobial composition according to claim 19, wherein the antimicrobial system comprises:
from about 0.01 to about 10% by weight of the composition of a surfactant selected from the group consisting of non-ionic surfactant, cationic surfactant, amphoteric surfactant and a mixture thereof;
from about 0.01 to about 10% by weight of the composition of a non-aqueous solvent; and
at least 50% by weight of the composition of water.
